(19) United States
(12) Patent Application Publication
(10) Pub. No.: US 2010/0292591 A1
(43) Pub. Date: Nov. 18, 2010

(54) Venous measurement system

(76) Inventors: Thom W. Rooke, Rochester, MN (US); David Liedl, Eyota, MN (US); William Davis, Uticia, MN (US)

Correspondence Address: Douglas L. Tschida, Suite 202, 93 Litttle Canada Road West, St. Paul, MN, 55117-2369, US

(21) Appl. No.: 12/800,453

(22) Filed: May 14, 2010

Related U.S. Application Data
(60) Provisional application No. 61/216,176, filed on May 14, 2009.

(51) Int. Cl. A61B 5/026

(52) U.S. Cl. 600/504

(57) ABSTRACT

A system and plethysmographic test procedure for evaluating venous blood volume/flow at a subject's legs. Occluding and sensing cuffs mounted to a subject's thighs and calves/ankles are inflated and deflated relative to controlled movement of a supporting chair. The chair provides hinged backrest, seat and footrest pieces and is controlled to manipulate the subject during several test phases as blood volume/flow is monitored. The chair is manipulated between a 1) baseline calibration position with the subject's back upright, seat horizontal to floor and feet lowered, 2) an outflow position with the subject's back tipped back flat and legs elevated above the heart, 3) a dependent filling position, rapid return of subject to back upright, seat horizontal to floor and feet lowered, and 4) an exercise position wherein the ankles are flexed with the back upright, seat horizontal to the floor and feet lowered. “Outflow”, “dependent filling”, “exercise” and “ejection fraction” flow values are determined relative to measured tracings exhibiting venous volume/flow changes in the legs.

### BACKGROUND OF THE INVENTION

The present invention relates to devices for evaluating peripheral vascular blood flow and, in particular, to a modular, occluding and sensing cuff system and cooperating chair/table assembly for evaluating venous blood flow in a subject's limbs (e.g. legs.)

The subject invention provides a system including a pivoting chair, two pneumatic cuff-based monitor assemblies and a central processor for conveniently and contemporaneously measuring venous blood flow/volume in a subject's legs relative to monitored pressures. The collected pressure data and derived values are used as one of many diagnostic aids to evaluate the vascular condition of a subject's legs.

### SUMMARY OF THE INVENTION

It is a primary object of the present invention to provide a low-cost, modular system for independently measuring and evaluating venous blood volume/flow in a subject's limbs.

It is a further object of the invention to provide a system for controlling the inflation and deflation of multiple cuff-based monitors mounted to each of a subject's legs and ankles relative to controlled movement of a test subject, for example back erect with legs lowered, back supine with legs elevated, and exercising motions of the subject limb (e.g. ankle flexes).

It is a further object of the invention to provide a chair comprised of backrest, seat and footrest pieces that pivot relative to each other and a supporting framework, independent cuff-based monitors fitted to multiple limbs of a seated subject, controls to manipulate the chair, and a processor for monitoring blood volume/flow through a test subject's legs relative to the inflation/deflation of occluding cuffs fitted to the subject's thighs and sensing cuffs fitted to the ankles and movement at the ankles.

It is a further object of the invention to provide a central processor, monitor and associated memory for controlling system operation and computing and displaying monitored and calculated parameters indicative of the venous condition of the subject's legs.

The foregoing objects, advantages and distinctions of the invention, among others, are found in a central processor based system that directs control and data transmissions to and from two monitor assemblies or sets of cuffs fitted to each of a subject's legs. Each monitor assembly includes an occlusion and a sensing cuff that independently communicate with the central processor/controller. The system generally performs a plethysmographic measurement of blood volume flow at each of a subject's legs relative to chair and leg movements in a matter of minutes.

Control and data signal communications occur over appropriate communication link(s) (e.g. hardwired, wireless or other appropriate communication path) between the central processor and the occlusion/sensing cuffs at each monitor assembly. Each monitor assembly generally operates independent of the other. Inflation and deflation control signals direct an air pump and cuff operation. During a “calibration period”, the sensing cuffs are each inflated in a stepwise fashion to establish a baseline condition and criteria. The occlusion cuffs are subsequently inflated to occlude blood flow through the legs and the chair is controlled to manipulate the subject's back to a supine position with the legs elevated. During a subsequent “outflow period”, the occluding cuffs are deflated and venous blood outflow is monitored via the sensing cuffs. During a “dependent filling” period, the chair and subject are next manipulated and returned to an upright condition and venous inflow or filling is monitored. During a final “exercise period”, venous flow is monitored as each leg is exercised (e.g. the ankles are flexed). A preprogrammed central processor/system controller continuously monitors venous blood flow volume during the test relative to pre-established test criteria to determine and report parameters and indicators useful to the evaluation of the subject's venous function and condition.

Still other objects, advantages and distinctions of the invention will become more apparent from the following description with respect to the appended drawings. Considered alternative assemblies, methodologies, improvements and/or modifications are described as appropriate. The description should not be literally construed in limitation of the invention. Rather, the scope of the invention should be broadly interpreted within the scope of the further appended claims.

### BRIEF DESCRIPTION OF THE DRAWINGS

Similar reference numerals and characters at the drawings refer to like structure at the various drawings and which are as follows:

FIG. 1 shows a generalized system diagram wherein two sets of inflatable occluding and sensing cuffs are fitted to a test subject's thighs and calves/ankles (only one set of which is shown) to monitor venous blood flow volume through the legs and communicate measured pressure data to a central processor.

FIG. 2 shows a system operational timeline relative to movement of associated chair piece arrangements and related venous blood flow volume measurements monitored by the system.

FIG. 3 shows an exemplary test result tracing indicative of monitored competence/function for patent flow (L, solid line) and obstructed flow (L1, dashed line) at a subject's left leg.

FIG. 4 shows an exemplary test result tracing indicative of monitored competence/function for patent flow (R, solid line) and obstructed flow (R1, dashed line) at a subject's right leg.

FIG. 5 shows a summary test result chart for the detected flows of FIGS. 3 and 4 relative to a pre-assigned 77% patent/obstructed ratio parameter, wherein the % ratios are computed from measured flow volumes 4 seconds after occluding cuff deflation÷maximum volume measured at the sensing cuff before and after deflation and plotted relative to the “y” axis.

FIG. 6 shows a test result tracing indicative of a monitored refill function for venous flow at a subject's left leg ((i.e. normal <5 ml/minute (solid line) and abnormal flow or >0.5 ml/minute (dashed line)).

FIG. 7 shows a test result tracing indicative of monitored refill function for venous flow at a subject's right leg ((i.e. normal <5 ml/minute (solid line) and abnormal flow or >0.5 ml/minute (LX, dashed line)).

FIG. 8 shows a test result tracing for the exercise period for normal (L, solid line) and abnormal (LX, dashed line) flow conditions indicative of monitored venous outflow function during the exercise period.

FIG. 9 shows a test result chart for the exercise period for normal (R, solid line) and abnormal (RX, dashed line) flow conditions indicative of monitored venous outflow function during the exercise period.

FIG. 10 shows a summary “ratio” test result chart for ratios determined from the detected flows of FIGS. 3, 4 and 8 and 9 relative to a pre-assigned 45% normal/abnormal ratio parameter, wherein the ratios are computed from the measured volume at the 90% level of the L and R refill volume tracings at the left and right legs depicted in FIGS. 8 and 9 (i.e. 1.1 normal and 0.5 abnormal) divided by the measured volume at the sensing cuff 4 seconds after occluding cuff deflation (i.e. 1.6 for L and R “patent” and 3.5 for LX and RX “obstructed flow”) conditions shown at FIGS. 3 and 4.

FIG. 11 shows an operational view of the system with a test subject (shown in dashed line) seated in an upright condition such as during the “calibration”, “passive draining or dependent fill”, and “exercise” periods.

FIG. 12 shows an operational view of the backrest lowered and the footrest elevated and which chair/subject condition is established during the “outflow” period.

FIG. 13 shows an operational view of the chair with the backrest, seat and footrest sections lowered, such as when the chair might be used as a conventional examination table.

FIG. 14 shows a pneumatic flow chart to the operation of an air pump and associated valves which inflate and deflate the occluding and sensing cuffs.

FIG. 15 shows a detailed schematic to the controller 30 of FIG. 1.

FIG. 16 shows a detailed schematic to the band pass filers 40 and 42.

FIG. 17 shows a detailed schematic to the pump driver circuitry.

### DESCRIPTION OF THE PREFERRED EMBODIMENT

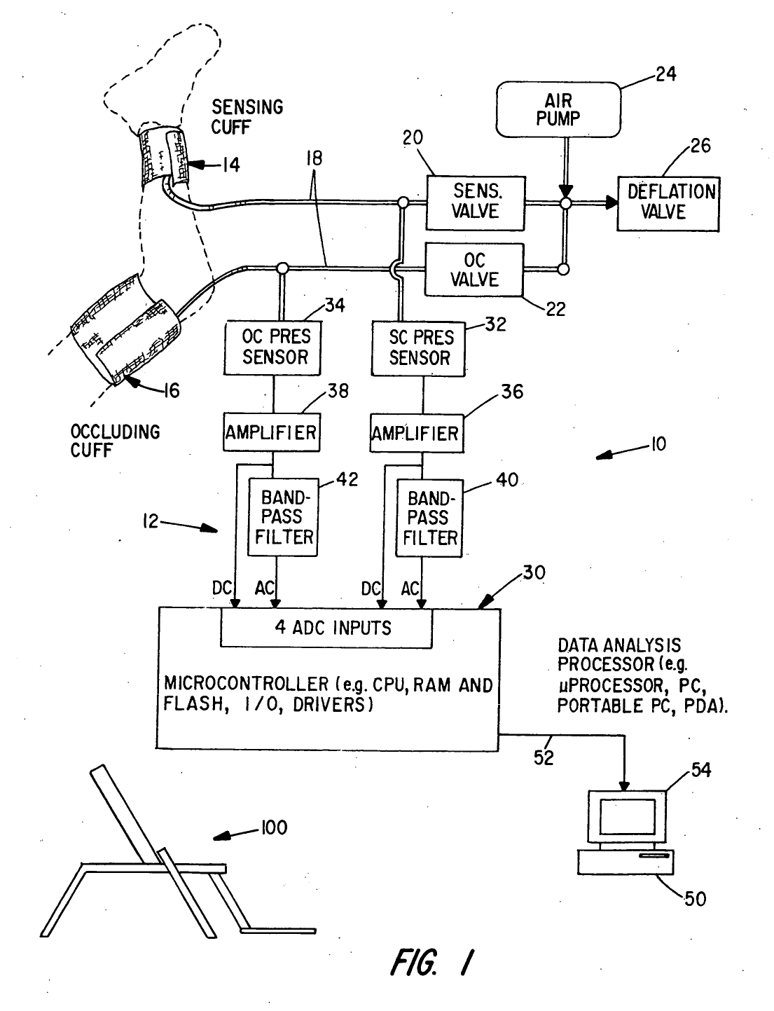

With attention to FIG. 1, a generalized overview is shown to the automatic venous blood flow measurement system 10 of the invention. The system 10 is depicted in relation to a chair 100 for manipulating a test subject and a single cuff monitor assembly 12 which is shown in an exemplary mounting to a test subject's thigh and calf/ankle. It is to be appreciated the system 10 when performing a test on a typical subject uses two identical monitor assemblies 12. One monitor assembly 12 is coupled to each of a subject's legs.

Each monitor assembly 12 comprises a pair of portable, inflatable sensing and occlusion cuffs 14 and 16 which are respectively constructed to perform sensing and occlusion functions. Typically, a sensing cuff 14 is fitted to the calf or ankle of a test subject and an occluding cuff is fitted to the subject's thigh. The cuffs 14 and 16 can be constructed to any desired shape and size to accommodate the limb and task to be performed. The cuffs 14 and 16 are cloth covered. The cuff 16 inflates and deflates over a nominal pressure range sufficient to occlude blood flow in the leg. The cuff 14 operates at pressures sufficient to retain the cuff 14 to the calf/ankle and maintain sensor contact with the leg.

The cuffs 14 and 16 are inflated and deflated via associated supply conduits 18, inflation valves 20 and 22, air pump 24 and deflation valve 26. The cuffs 14 and 16 include appropriate fasteners (e.g. overlapping hook and loop fasteners) to securely attach to a limb (e.g. upper arm, leg or ankle) or appendage (e.g. wrist, finger or toe). Presently, the sensing cuffs 14 are constructed to be slightly smaller than the occluding cuffs 16 to facilitate attachment to the distal sensing regions of the legs (e.g. calves or ankles). A cuff manufactured by the Hokanson Co. is presently used as the occluding cuff 16 and a CRITIKON™ cuff manufactured by General Electric Co. is used for the sensing cuff 14.

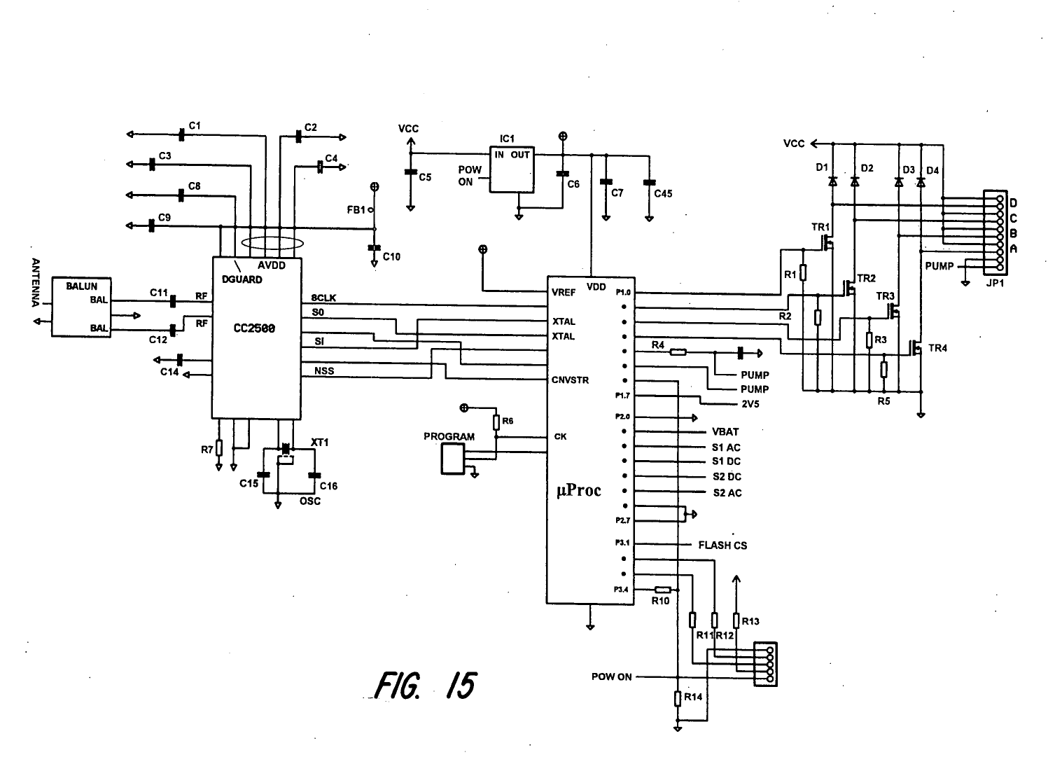

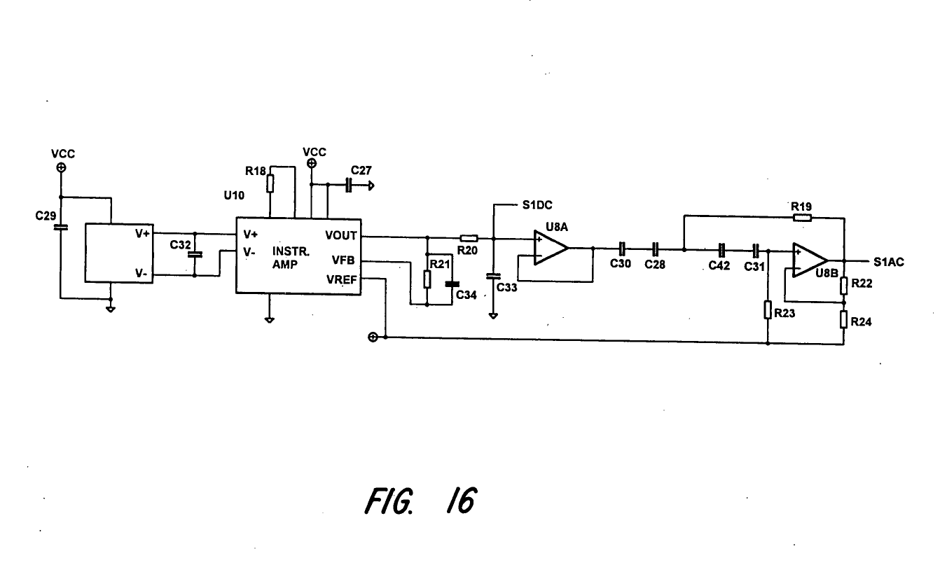

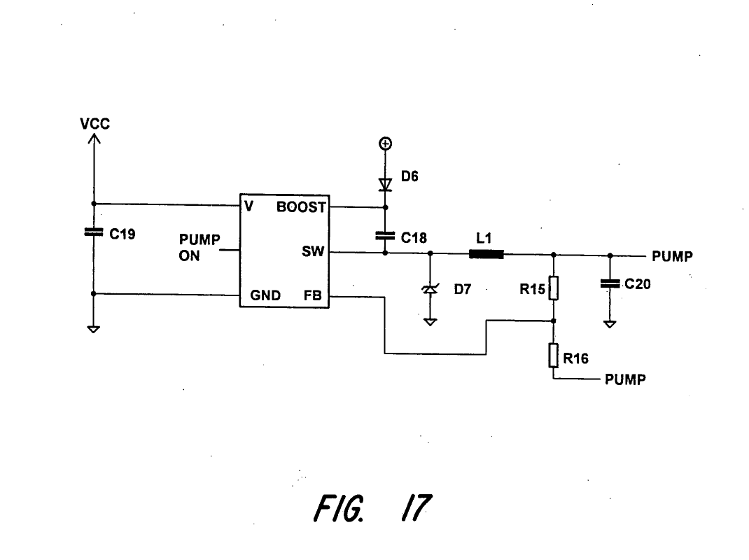

Each set of portable sensing and occluding cuff assemblies 14 and 16 are coupled to monitor control circuitry 30 (i.e. monitor controller, shown in a detailed schematic at FIG. 15) via intermediate sensors 32, 34, amplifiers 36, 38 and filters 40, 42 (e.g. band pass filters, shown in detailed schematics at FIG. 16). The sensors 32, 34 are incorporated into the cuffs 14 and 16. Upon inflation of the associated cuffs 14 and 16, the sensors 32, 34, detect and produce electrical signals indicative of sensed pressures. The detected signals are amplified and the components are coupled to appropriate input ports 44 of the controller 30.

The sensors 32, 34 can be constructed from any of a variety of devices that can sense changes in a physical condition and produce a related electrical signal. For example, piezoelectric elements, strain gauge or optical assemblies are able monitor and convert physical movements at the subject to electrical signals. Preferably any selected pressure measuring device is adaptable to a cuff mounting.

The monitor controller circuitry 30 includes a processor unit (e.g. microprocessor/CPU), associated storage memory (e.g. RAM, ROM, flash) of suitable type and configuration, drivers and input/output (I/O) circuitry to communicate with a primary or central processor 50. The controller 30 responds to a preprogrammed or programmable instruction set to control the operation of each monitor assembly 12 relative to the air pump and monitor sensed pressures.

The backrest, seat and footrest sections of the chair are independently controlled via associated pneumatics and the functions of which are described below and shown at the pneumatic flow chart of FIG. 14. A detailed schematic to the controller 30 is shown at FIG. 15. A detailed schematic to the band pass filers 40 and 42 are shown at FIG. 16 and a detailed schematic to the pump driver circuitry is shown at FIG. 17.

The monitor controller 30 also includes I/O circuitry that communicates over a suitable communications link 52 (e.g. hardwired conductors, wired network, phone system, wireless system, WIFI etc.) with the primary processor 50. The processor 50 is coupled to a display monitor 54 or other suitable device that displays and communicates relevant test information to an operating technician (e.g. CRT, LCD/TFT screen and/or printer).

Depending upon the system 10, the processor 50 can comprise a portable or stationary PC, a mid-sized or mainframe central computer, PDA, special purpose handheld device or any other device containing an appropriately programmed processor with supporting storage memory, communications capabilities and sundry other devices. The processor 50 is operative to perform the necessary interpolation of the test data and display the operation of each monitor 12 and the results obtained for each diagnostic test at the monitor 54.

The processor 50 presently comprises a portable, laptop computer with suitable processing power and communications capabilities. Presently a hardwired link 52 is established with each of the cuff monitor assemblies 12. The processor 50 might also be coupled to the monitors 12 via other wired or wireless network or internet connections and/or to other larger systems where the test data is stored in a database.

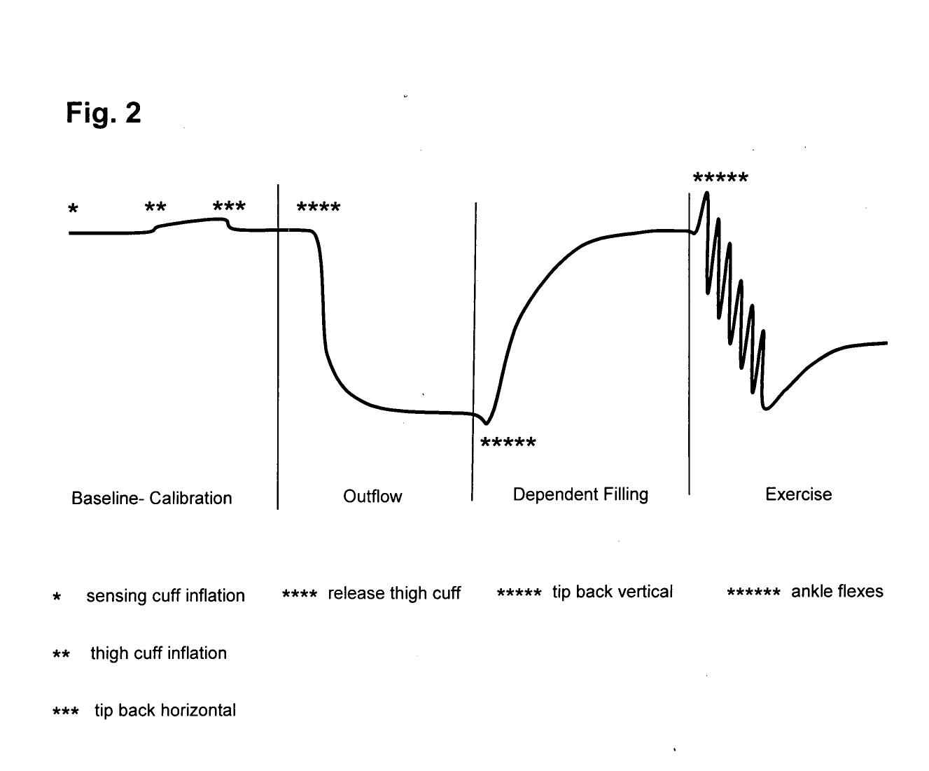

With attention next directed to FIGS. 2 through 10, the system 10 provides an automatic, plethysmographic method and sequence of steps that are performed during a typical venous test procedure which are as follows:

### I. Baseline—Calibration Phase:

A. Arrange Test Subject

Arrange test subject in an upright seated position as shown at FIG. 11 relative to pneumatically controlled chair 100. The chair 100 provides a padded backrest 102 and padded seat 104 and a footrest 106. The back rest 102 and footrest pieces 102 and 106 are coupled to associated pneumatic and manual controls to selectively manipulate the subject during the test. A foot controller 108 and associated pedal switches 110 operate to control the rotation of the backrest 102 and footrest 106.

B. Mount Sensing Cuffs 14 (e.g. Calf or Ankle)

Measure the circumference of each leg 6 mm above the medial malleolus and record in mm. The measurements are typically entered in the computer at “venous” test program “circumference prompts”.

Position the sensing cuffs 14 around both legs. Place the cuffs 14 loosely to the left and right legs (e.g. with a two finger space the between cuff and calf). The bottom of each cuff 14 is located to nominally rest on the top of the foot.

Connect the tubing 112 that extends from the sensing cuffs 14 to left and right sensing cuff tubing ports at the controller 114 on the chair 100.

C. Mount Occluding Cuffs 16 (e.g. Thigh)

Position each occluding cuff 16 around each thigh. Each cuff 16 is again loosely fitted with a two finger space between the cuff and the thigh.

Connect the tubing 112 that extends from the occluding cuffs 16 to left and right occluding cuff tubing ports 118 and 120 on the chair 100.

D. Calibration (i.e. Measure Pressure/Volume Relationship at Sensing Cuffs 14)

The calibration process begins when the “start” button is selected. The air inflation sequence begins by first inflating the left sensing cuff 14, then the right sensing cuff 14, each to a nominal pressure of 2.5 mmHg. The air inflation sequence continues by pumping more air into each cuff 14. During this step, air is first admitted into the left sensing cuff 14 and then the right sensing cuff 14 until each cuff is pressurized to cuff pressure of 5 mmHg. As the sensing cuffs 14 are inflated, the volume of air needed to increase the pressure from 2.5 mmHg to 5 mmHg is measured at each sensing cuff to establish a pressure/volume relationship.

E. Baseline:

Once the air inflation process is completed, active pressure versus time in seconds tracings are displayed on the monitor screen 54 for the sensing cuffs 14 at the left and right legs (e.g. left—red and right—green). A baseline is reached when a predetermined pressure value programmed into a “setting” or test criteria parameter section of the controller program is reached.

Once a stable baseline condition is confirmed, the thigh cuffs 16 are inflated and held at 60 mmHg. At this time, the sensing cuff tracings will rise due to blood being trapped in the extremity. After 4 seconds following thigh cuff inflation, the computer 50 produces an operator prompt “Tip patient back and press “OK”” to appear at the monitor 54. The subject is then tipped back in the seated position until the backrest 102 is horizontal to the floor and the subject's legs are positioned above the level of the heart. The subject is now positioned to begin “outflow plethysmography” with the object of looking for indicators of venous obstruction.

### II. Outflow Plethysmography Period (Objective—Are There Obstructions or Blockages in the Veins of the Leg?)

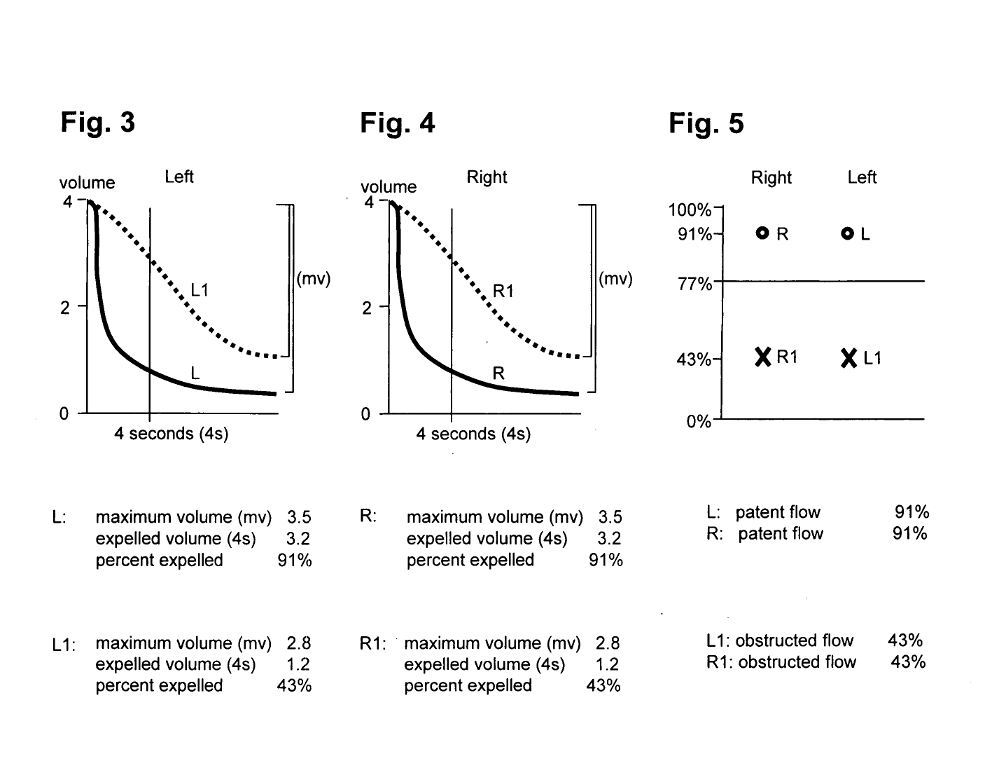

With attention to FIGS. 2 and 3 through 5 and four seconds after pressing “OK”, the occluding cuffs 16 deflate completely. This causes the blood trapped in the lower leg to rush downward towards the heart. The sensing cuffs 14, in turn, continuously monitor and measure the amount of venous blood in the lower leg during and after the deflation period. The data is displayed at the tracings shown at FIGS. 3 and 4 and wherein a patent or normal flow condition is shown at the solid line tracings L and R and an obstructed flow condition is shown at the dashed line tracings L1 and R1.

Data Points of Interest

Volume measurements on the left and right sensing cuff 14 tracings are taken at a predetermined time of 4 seconds after deflating the occluding cuff 16. A ratio is then calculated for each leg by the processor 50 by dividing the volume at 4 seconds by the maximum volume from the L and R tracings.

A maximum flow volume is also obtained for the left and right legs by measuring the volume in the respective sensing cuffs 14 before and after deflation.

Evaluation Per Pre-Established Test Criteria

With attention to FIG. 5, the flow at a limb that is deemed “patent” if a ratio value greater than or equal to 77% is obtained. Flow at a limb is deemed “obstructed” if a ratio value less than 77% is obtained.

By way of an analogy to water flow versus blood flow, this test phase acts to dam the water at the occluding cuff before allowing the dam to release the water. The water then rushes out and, if the water path is open, the water is free flowing. If there are blockages in the water course, the flow is slowed and the water's path is considered obstructed.

Conversely, once the occluding pressure on the L and R occluding cuffs 16 is released, the venous blood trapped in the lower legs is able to move freely towards the heart limited only by the condition of the venous system and presence of any obstructions. If the blood can move freely, the volume in the sensing cuff decreases quickly (i.e. demonstrating “patent” flow). If the blood cannot move freely, the volume in the sensing cuff goes out slowly (i.e. demonstrating “obstructed” blood flow).

III. Dependent Venous Filling Period (Objective—Enable Dependent Filling to Measure Valve Incompetence by Testing How Fast the Legs Fill Up with Venous Blood?)

With attention to FIGS. 2, 6 and 7 and at the beginning of this test period, the subject's legs are initially held higher than the level of the heart and empty of venous blood. The period is initiated with the monitor 54 displaying a prompt “Press “OK”” causing the operator to engage and hold the appropriate pedal switch 110 to a “fast” position to bring the patient forward to an upright condition. The test subject is quickly brought forward while the sensing cuffs 14 measure the volume of blood that responsively flows into the lower leg.

Data Points of Interest

A volume value measured by the L and R sensing cuff tracings are determined at 7.5 seconds.

A maximum value is obtained by measuring the volume in the L and R sensing cuffs 14 before and after bringing the patient forward.

Evaluation Per Pre-Established Test Criteria

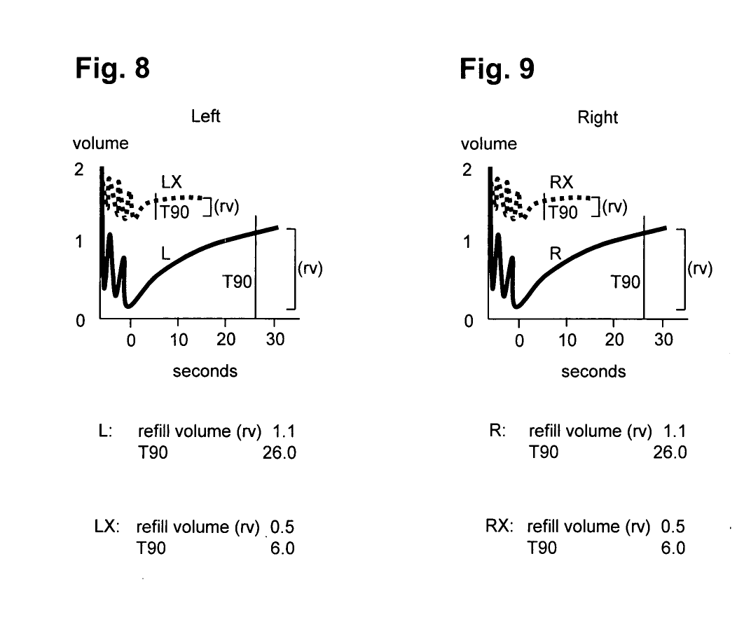

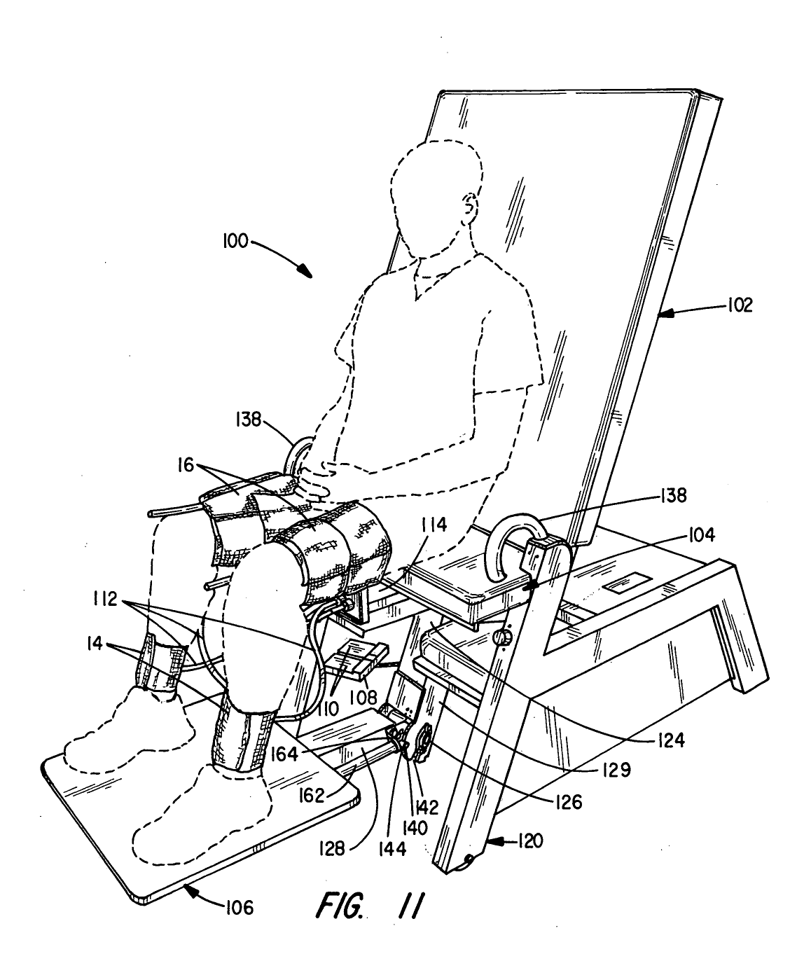

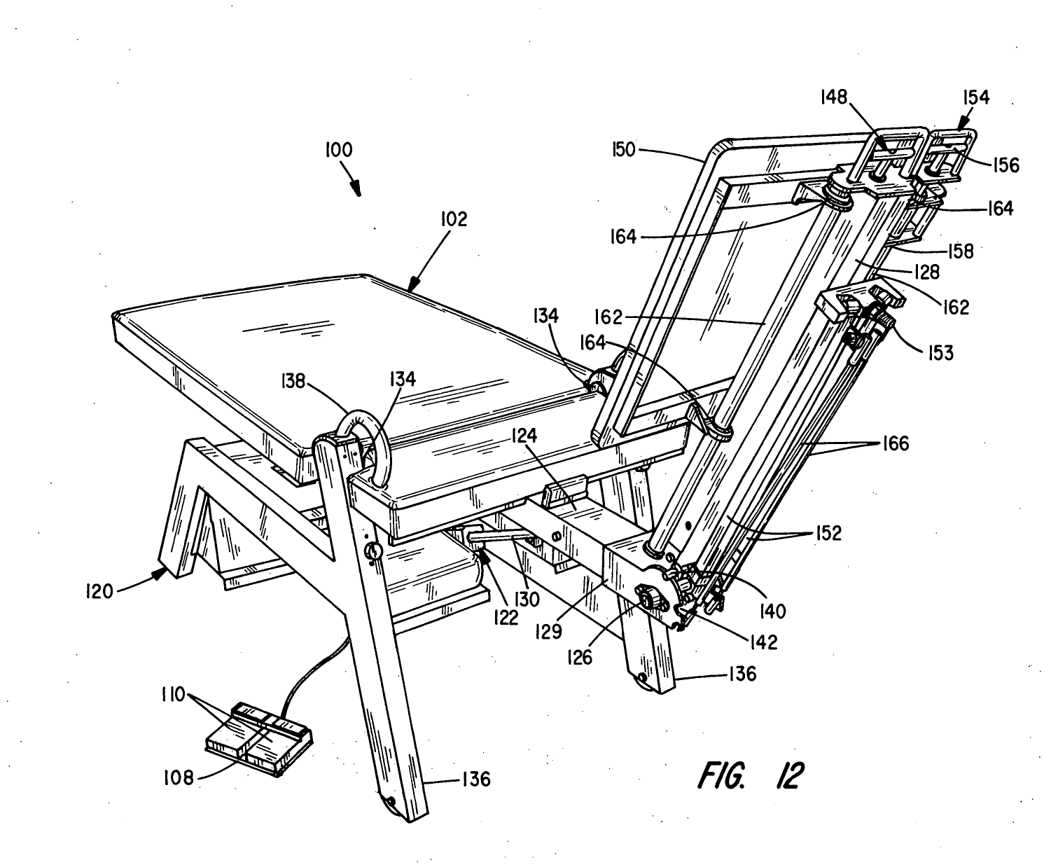

With attention to FIGS. 8 and 9, measure L and R re-fill rate or flow volumes at 7.5 seconds after establishing the subject in an upright condition (*8=ml/min). Blood flow is to be deemed normal if measured value is <5 ml/minute and abnormal if the flow is >5 ml/minute.

By way of analogy and at the beginning of the dependent filling test period, the legs are higher than the level of the heart and empty of blood. When the patient is quickly brought forward, the venous blood attempts to rush back into the lower legs. Valves present in the venous system however prevent the blood from freely rushing back into the legs. In a limb that has normal valve control, the rate of refilling is very slow or usually less than 5 ml/minute. In a limb with damaged valves, the rate of refilling is very fast or usually more than 5 ml/minute.

IV. Venous Exercise Period (Objective—Measure Venous Function or How Much Venous Blood can be Pumped with Ankle Flexes and Calf Muscle Pump Action?)

With attention to FIGS. 2, 8 and 9 and at the beginning of this test period, the subject is upright and the legs are full of venous blood. The L and R sensing cuffs 14 are mounted to responsively continue to measure the amount of venous blood flow in the left and right lower legs. The period is initiated with the monitor 54 displaying a prompt for the subject to perform 10 ankle flexes. Following the 10 ankle flexes, the subject remains still while the venous blood is allowed to refill with venous blood.

Data Points of Interest

A volume value is obtained from the L and R sensing cuff tracings when 90% of the maximum volume returns to the legs.

A maximum volume value is also obtained by measuring the volume at the L and R sensing cuffs 14 before and after ankle flexes.

Evaluation Per Pre-Established Test Criteria

Normal and abnormal blood flow conditions are determined in relation to the point in time or T90 when 90% of maximum volume returns to the legs. A normal condition is indicated if a time >25 seconds is determined and an abnormal condition is indicated if a time <25 seconds is determined.

By way of a generalized summary and during the exercise test period, the legs are full of venous blood at the beginning of the exercise test period. As the ankles are flexed blood is normally pumped out of the lower leg to the heart. After the ankle flexes, the venous blood pumped out attempts to rush back into the lower legs. Valves in the venous system however again prevent the blood from rushing back into the legs for a period that normally exceeds 25 seconds. An abnormal result is obtained if 90% of the blood is allowed to pass back into the legs in less than 25 seconds due to faulty valves.

V. Ejection Fraction (Objective—Combine Data from Dependent Filling and Exercise Periods:

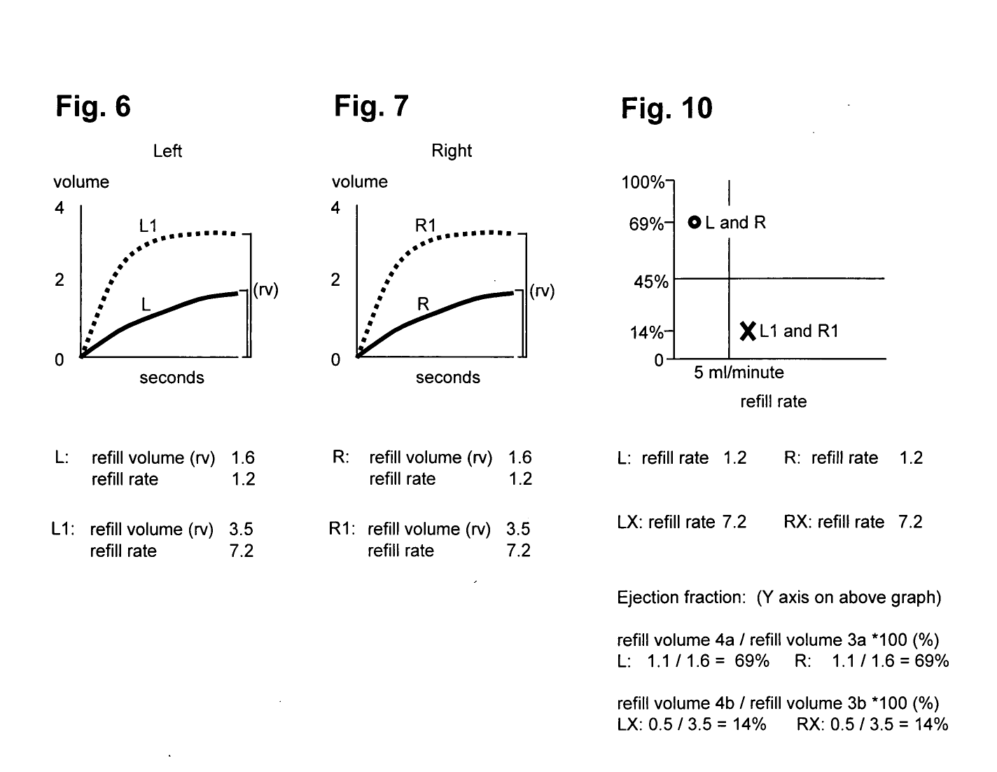

With attention to FIG. 10, a “venous ejection fraction” is calculated by dividing the maximum volume measured at the venous exercise period (FIGS. 8 and 9) by the maximum volumes measured during the dependent venous filling periods (FIGS. 6 and 7). The resulting L and R and L1 and R1 ratios are depicted at FIG. 10. A normal condition is deemed to occur for a ratio >50% and an abnormal condition is deemed to occur for a ratio <50%.

Again and by way of a generalized overview of the “ejection fraction”, if the dependent venous filling maximum volume value is deemed to exhibit a “full tank” and the venous exercise maximum volume is deemed to represent the amount of blood pumped from the lower legs during exercise. The ratio defines how much blood was pumped during leg exercise when each leg has its own maximum volume.

Returning attention to FIGS. 11 through 13, the backrest, seat and footrest pieces 102, 104 and 106 of the chair 100 are mounted to rotate relative to a surrounding framework 120 to support the foregoing venous test procedure. A pneumatically controlled “acme screw” piston assembly 122 is particularly secured to rotate a longitudinally centered backrest support frame member 124 that is mounted to pivot relative to the framework 120. An axle 126 secures a footrest support frame member 128 to a bracket 129 secured to the end of the backrest support frame member 124. The footrest support member 128 is mounted to rotate about the axle 126 between upper and lower latching notches 140 and 142. The screw/piston assembly 122 is controlled via the foot controller 108 and associated pedal switches 110.

The backrest support frame member 124 is separately mounted to rotate about left and right pivot assemblies 134 fitted to the lateral sides of the backrest 102 and upright frame members 136 at the framework 120 adjacent handgrips 138. Limit switches (not shown) associated with the pivot assemblies 134 and the backrest support member 124 and framework 120 monitor and control the rotation angle of the backrest 102 relative to the seat 104.

As a piston 130 at the assembly 122 is retracted, the backrest support member 124 is drawn downward which pivots the backrest support member 126 and backrest 102 to an upright condition. As the piston 130 is extended, the backrest support member 124 is raised which pivots the backrest support member 124 and backrest 102 to a supine, horizontal condition and coplanar with the seat 104.

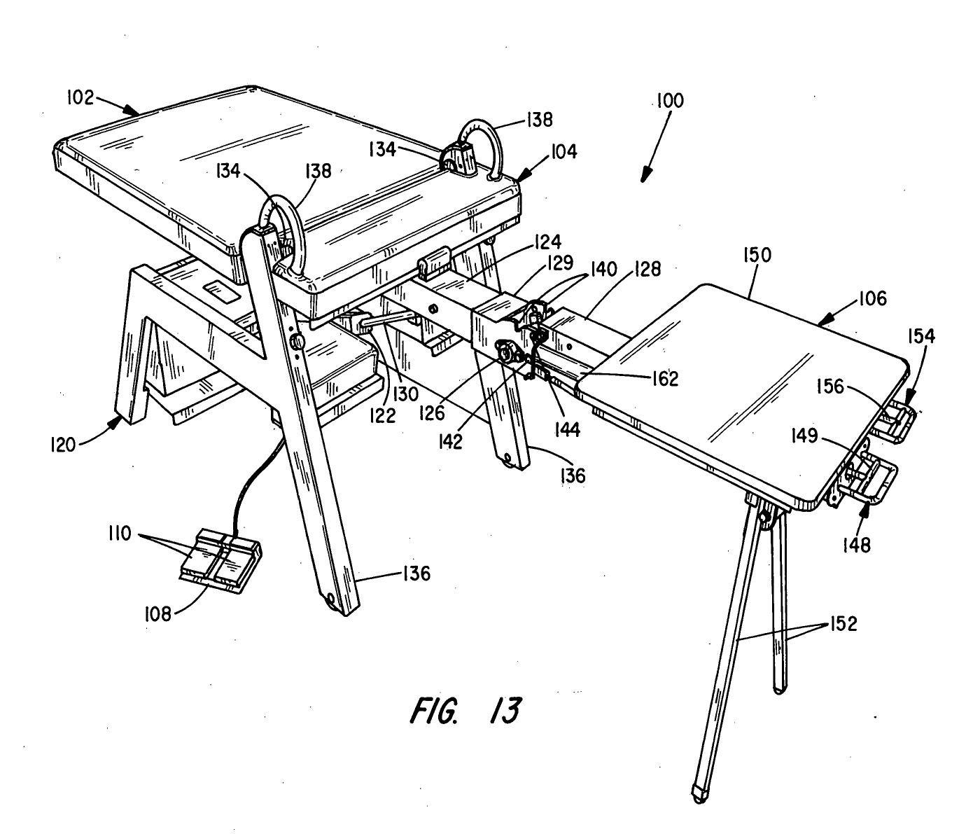

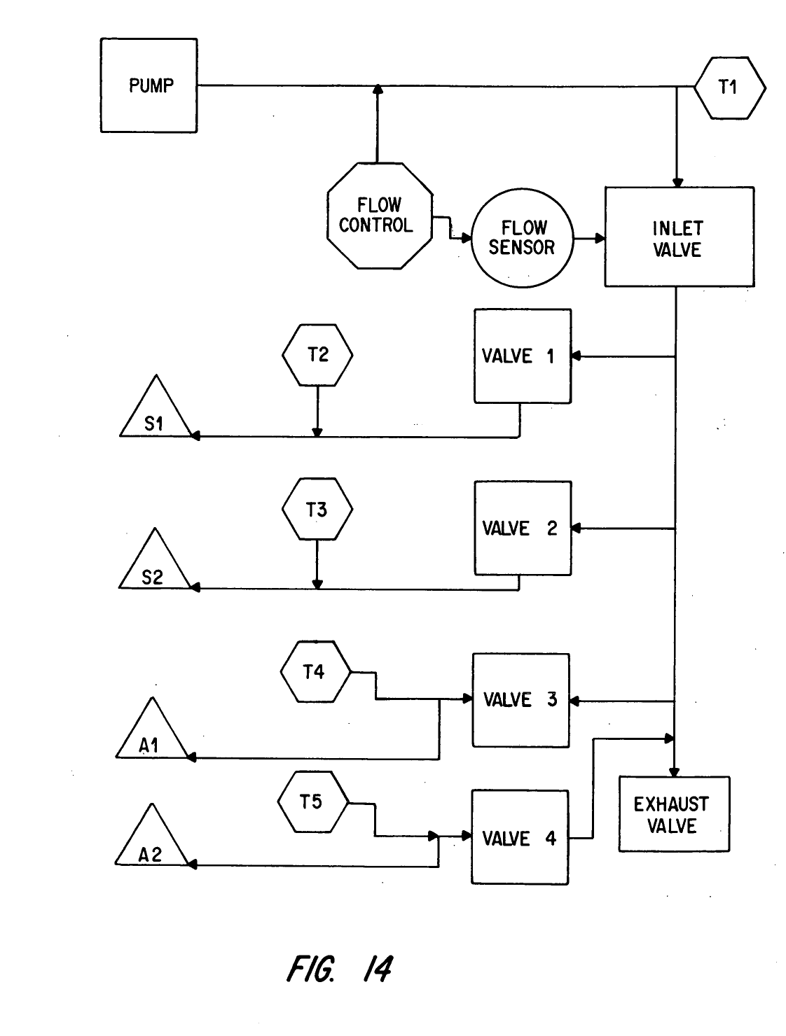

As the backrest 102 moves between the upright and supine conditions, the footrest 106 is maintained at a constant angle (e.g. 90 to 115 degrees) relative to the backrest 102 and simultaneously rotates between a lowered horizontal condition parallel to the seat 104 and an elevated transverse condition relative to the coplanar seat 104 and backrest 102, see FIGS. 11 and 12. The notches 140 at the bracket 129 secured to the backrest support member 124 cooperate with manually controlled, spring biased latch pins 144 that laterally extend from the footrest support frame member 128 to fix the angular orientation of the footrest 106 at the depicted orientations. The lower notches 142 as discussed below with respect to FIG. 13 facilitate a further horizontal coplanar mounting orientation of the footrest 106 with the backrest 102 and seat 104.

With attention to FIG. 13, an additional horizontal support orientation of the footrest 106 is depicted. In this orientation, the backrest, seat and footrest pieces 102, 104 and 106 are collectively arranged to lie in a coplanar horizontal, supine mounting orientation. This horizontal orientation allows a subject to lie completely flat. The multiple positions shown at FIGS. 11-13 thereby facilitate use of the chair 100 at a medical facility to perform the foregoing venous testing and as a general examination table.

The horizontal footrest orientation is obtained by manually lowering the footrest 106 from the elevated transverse condition shown at FIG. 12 to the horizontal condition shown at FIG. 13. A latch pin release assembly 148 secured to the bottom of a footrest platform 150 is particularly used to release the latch pins 144 from the notches 140 to permit the rotation of the footrest support member 128 and platform 150 to a horizontal condition. Once the member 128 and platform 150 are horizontal condition the latch pins 144 align with and are released to engage the notches 144.

As the footrest platform 150 is rotated, support legs 152 arranged in a bi-pod fashion are separately released with a hand knob 153 to pivot and extend orthogonal to the footrest support member 128 and footrest platform 150.

The displacement of the footrest platform 150 from the seat 104 is separately adjusted with a separate latching assembly 154. The latch assembly 154 comprised of a handle 156, cable 158 and spring-finger release mechanism (not shown) that grips a slide rail 162 to which the footrest platform 150 is mounted. The platform 150 is particularly mounted to slide along a pair of rails 162 via associated slide bearings 164. Upon pulling the handle 156, the release releases its grip on the slide rail 162 which allows the platform 150 to be moved to and fro relative to the seat 104. Control arms 166 and pneumatic pistons (not shown) mounted in a channel at the backrest support member 124 control and resiliently bias the rotation of the footrest platform 150 and legs 152 to prevent a freefall situation.

By way also of a summary of the operation of the pneumatic flow chart of FIG. 14 relative to the control of the cuffs 14 and 16 during the foregoing test procedure, the pneumatics operate as follows:

1) pump runs until T1 reads 2 bar (pump turn on/off to keep line pressure at 2 bar);

2) inlet valve shifts to let air flow through the flow sensor through valve 1 to S1 until T2 reads 3 mm/hg;

3) inlet valve shifts to let air flow through the flow sensor through valve 2 to S2 until T3 reads 3 mm/hg;

4) inlet valve shifts to let air flow through the flow sensor through valve 1 to S1 until T2 reads 6 mm/hg;

5) inlet valve shifts to let air flow through the flow sensor through valve 2 to S1 until T3 reads 6 mm/hg;

6) inlet valve shifts to bypass flow sensor, air flows through valve 3 to A1 until T4 reads 60 mm/hg;

7) inlet valve shifts to bypass flow sensor, air flows through valve 4 to A2 until T5 reads 60 mm/hg;

8) Step 6 is cycled to maintain 60 mm/hg in A1 and A2;

9) Pump is turned off;

10) valve 3 and exhaust valve open to dump pressure from A1 and A2;

11) valve 1, valve 2 and exhaust valve open to dump pressure from S1 and S2.

The backrest 102, seat 104 and footrest 106 are independently manipulated via the pneumatic screw assembly 122, foot controller 108 and switches 110.

While the invention has been described with respect to a preferred system assembly and sequence of test steps, alternative system arrangements, test protocols and procedures, and chair configurations can be developed. Alternative sequences and manners of conducting other venous blood flow measurements can also be performed or may be suggested to those skilled in the art. The scope of the invention should therefore be construed broadly within the spirit and scope of the following claims.

What is claimed is:

1. Apparatus for evaluating venous blood flow comprising:
a) a chair including a framework supporting a backrest, a seat and a footrest and wherein said backrest and footrest are mounted to pivot relative to said seat;
b) an air pump;
c) deflation means;
d) a pressure monitor adapted to be coupled to a leg of a test subject, wherein the monitor comprises 1) inflatable occlusion and sensor cuffs coupled to said air pump and said deflation means and wherein said sensor cuff includes a pressure sensor and 2) means for controlling i) said air pump to inflate said sensor cuff to establish at least two pressure levels to determine a baseline condition relative to air volume, ii) said air pump to inflate said occlusion cuff to occlude blood flow through the leg, and iii) said deflation means to deflate said occlusion and sensor cuffs;
e) means for positioning said chair between a first condition wherein said backrest extends transverse to said footrest and seat and said seat and footrest are vertically displaced apart in parallel relation to each other and a second condition wherein said backrest and said seat align in coplanar horizontal alignment and said footrest transversely extends above said seat and backrest; and
f) means coupled to monitor and visually display venous blood flow pressures continuously sensed by said sensor cuff at said first and second chair conditions relative to inflated and deflated conditions of said occlusion cuff and to leg movements and display time based blood flow volume tracings and % ratios determined from said tracings indicative of the vascular condition of the monitored leg.

2. Apparatus as set forth in claim 1 wherein occlusion and sensor cuffs of a second pressure monitor are coupled to a second leg of the subject.

3. Apparatus as set forth in claim 1 wherein said footrest is supported to said backrest to pivot to a third condition wherein said backrest, seat and foot rest are coplanar to each other.

4. Apparatus as set forth in claim 3 wherein said backrest includes a first support member, wherein said footrest includes a second support member and wherein said first and second support members are mounted to pivot relative to each other to establish said first, second and third chair conditions.

5. Apparatus as set forth in claim 4 wherein said footrest comprises a platform mounted to move to and fro relative to said second support.

6. Apparatus as set forth in claim 5 wherein said footrest comprises a leg member mounted to pivot between parallel and orthogonal orientations to said platform whereby the footrest platform is supported to a chair support surface at said third chair condition.

7. Apparatus as set forth in claim 4 wherein including means for biasing the rotation of said footrest to prevent freefall.

8. Apparatus for evaluating venous blood flow comprising:
a) a chair including a framework supporting a backrest, a seat and a footrest and wherein said backrest and footrest are mounted to pivot relative to said seat;
b) an air pump;
c) deflation means;
d) first and second pressure monitors respectively adapted to be coupled to a leg of a test subject, wherein each pressure monitor comprises 1) inflatable occlusion and sensor cuffs coupled to said air pump and said deflation means and wherein said sensor cuff includes a pressure sensor, and 2) means for controlling i) said air pump to inflate said sensor cuff to establish at least two pressure levels to determine a baseline condition relative to air volume, ii) said air pump to inflate said occlusion cuff to occlude blood flow through the leg, and iii) said deflation means to deflate said occlusion and sensor cuffs;
e) means for positioning said chair between a first condition wherein said backrest extends transverse to said footrest and seat and said seat and footrest are vertically displaced apart in parallel relation to each other and a second condition wherein said backrest and said seat align in coplanar horizontal alignment and said footrest transversely extends above said seat and backrest; and
f) means coupled to monitor and visually display venous blood flow pressures continuously sensed by said sensor cuffs at said first and second chair conditions relative to inflated and deflated conditions of said occlusion cuffs and to leg movements and display time based volume tracings and % ratios determined from said tracings indicative of the vascular condition of the monitored legs.

9. A method for determining a venous blood flow condition of a subject comprising:
a) positioning a test subject in an upright condition in a chair including a framework supporting a backrest, a seat and a footrest, wherein said backrest and footrest are mounted to pivot relative to said seat, and wherein at said upright condition said backrest extends transverse to said footrest and seat and said seat and footrest are vertically displaced apart in parallel relation to each other coplanar to said seat and said footrest;
b) coupling a pressure monitor to a leg of a test subject comprising a first inflatable cuff coupled to a thigh and a second inflatable cuff having a pressure sensor coupled to the calf and operative to sense blood flow;
c) inflating said second cuff to first and second pressures to establish a baseline condition;
d) inflating said first cuff to occlude blood flow through the leg;
e) pivoting said backrest coplanar to said seat and said footrest to elevate said footrest and leg above the heart of said subject;
f) deflating said first cuff and monitoring blood outflow at said leg to determine an outflow volume indicative of normal or abnormal flow relative to a first criteria;
g) pivoting said backrest to said upright condition and monitoring return blood flow to said leg to determine a re-filling volume indicative of normal or abnormal blood flow relative to a second criteria; and
h) exercising said leg at said upright condition to pump a predetermined amount of blood from said leg and monitoring the subsequent return blood flow to said leg to determine a return flow volume indicative of normal or abnormal flow relative to a third criteria.

10. A method as set forth in claim 9 including determining a fraction comprising the maximum return flow volume monitored after exercise divided by a maximum re-filling volume monitored upon returning said chair to said upright condition to determine a ratio indicative of normal or abnormal flow relative to a fourth evaluation criteria.

11. A method for determining a venous blood flow condition of a subject comprising:
a) positioning a test subject in an upright condition in a chair including a framework supporting a backrest, a seat and a footrest, wherein said backrest and footrest are mounted to pivot relative to said seat, and wherein at said upright condition said backrest extends transverse to said footrest and seat and said seat and footrest are vertically displaced apart in parallel relation to each other coplanar to said seat and said footrest;
b) coupling first and second pressure monitors to the legs of a test subject respectively comprising a first inflatable cuff coupled to a thigh and a second inflatable cuff having a pressure sensor coupled to the calf and operative to sense blood flow;
c) inflating the second cuffs of said first and second pressure monitors to first and second pressures to establish a baseline condition;
d) inflating said first cuffs of said first and second pressure monitors to occlude blood flow through the legs;
e) pivoting said backrest coplanar to said seat and said footrest to elevate said footrest and legs above the heart of said subject;
f) deflating said first cuff and monitoring blood outflow at said legs to determine an outflow volumes indicative of normal or abnormal flow relative to a first criteria;
g) pivoting said backrest to said upright condition and monitoring return blood flow to said legs to determine re-filling volumes indicative of normal or abnormal blood flow relative to a second criteria; and
h) exercising said legs at said upright condition to pump a predetermined amount of blood from said legs and monitoring the subsequent return blood flow to said legs to determine return flow volumes indicative of normal or abnormal flow relative to a third criteria.

12. A method as set forth in claim 11 including determining a fraction for each leg comprising the maximum return flow volume monitored after exercise divided by a maximum re-filling volume monitored upon returning said chair to said upright condition to determine ratios indicative of normal or abnormal flow at each leg relative to a fourth evaluation criteria.
